(19) United States
(12) Patent Application Publication
(10) Pub. No.: US 2005/0020863 A1
(43) Pub. Date: Jan. 27, 2005

(54) Method of making fluorinated propanes

(75) Inventors: Hsueh Sung Tung, Getzville, NY (US); Kevin D. Uhrich, Alden, NY (US)

Correspondence Address: Colleen D. Szuch, Esq.;Honeywell International Inc., 101 Columbia Road-Patent Department, Morristown, NJ, 07962-2245, US

(73) Assignee: Honeywell International Inc.

(21) Appl. No.: 10/627,018

(22) Filed: Jul. 25, 2003

(51) Int. Cl. C07C 19/00

(52) U.S. Cl. 570/164

(57) ABSTRACT

The invention provides a process for the manufacture of fluoropropanes, and more particularly, the manufacture of 1,1,1,3,3,3-hexafluoropropane (HFC-236fa) and 1,1,1,2,3,3,3-heptafluoropropane (HFC-227ea). The process utilizes 3-carbon by-products, i.e. waste material, from other commercial processes as raw material. The process also avoids the use of hexafluoropropane (HFP) as a reactant for making HFC-227ea, and is able to convert any three-carbon hydrocarbon (HC), hydrochlorofluorocarbon (HCFC), chlorofluorocarbon (CFC) compound or any halogenated propanes and produce high valued three-carbon hydrofluorocarbons (HFCs) at significantly lower cost than current commercial processes.

### BACKGROUND OF THE INVENTION

The present invention relates to a process for the manufacture of fluoropropanes, and more particularly, the manufacture of hydrofluorocarbons or hydrochlorofluorocarbons. More particularly, the invention pertains to a process for the manufacture of 2,2-dichloro-1,1,1,3,3,3-hexafluoropropane (CFC-216aa), 2-chloro-1,1,1,2,3,3,3-heptafluoropropanes (CFC-217ba), 1,1,1,3,3,3-hexafluoropropane (HFC-236fa), and 1,1,1,2,3,3,3-heptafluoropropane (HFC-227ea). The invention describes an inexpensive process for the manufacture of the above-mentioned fluorinated propanes using a mixture of three-carbon reactants which may be obtained as waste streams from a variety of processes. The CFC-216aa and CFC-217ba can be used as intermediates for making HFC-236fa and HFC-227ea, respectively. They can also be used as intermediates for the preparation of other fluorinated compounds.

Traditionally, chlorofluorocarbons (CFCs) like trichlorofluoromethane and dichlorodifluoromethane have been used as refrigerants, blowing agents and diluents for gaseous sterilization. In recent years, there has been universal concern that completely halogenated chlorofluorocarbons might be detrimental to the Earth's ozone layer. Consequently, there is a worldwide effort to use fluorine-substituted hydrocarbons which contain fewer or no chlorine substituents. Accordingly, the production of hydrofluorocarbons (HFC's), or compounds containing only carbon, hydrogen and fluorine, has been the subject of interest to provide environmentally desirable products for use as solvents, blowing agents, refrigerants, cleaning agents, aerosol propellants, heat transfer media, dielectrics, fire extinguishing compositions and power cycle working fluids.

Two compounds in particular, 1,1,1,3,3,3-hexafluoropropane (i.e. CF₃CH₂CF₃ or HFC-236fa) and 1,1,1,2,3,3,3-heptafluoropropane (i.e. CF₃CHFCF₃ or HFC-227ea), are particularly suitable for such uses. HFC-236fa and HFC-227ea have zero ozone depleting potential, relatively low global warming potential and relatively short atmospheric lifetime. It is particularly useful where an environmentally acceptable agent is essential, where clean-up of other media presents a problem, where weight versus suppression potential is a factor, and where an electrically non-conductive medium is needed. HFC-236fa and HFC-227ea are odorless, colorless, liquefied compressed gas. Each one is stored as a liquid and dispensed as a colorless, electrically non-conductive vapor that is clear and does not obscure vision. They leave no residue and have acceptable toxicity for use in occupied spaces. HFC-227ea is capable of extinguishing a fire by a combination of chemical and physical mechanisms. Furthermore, it does not displace oxygen and therefore is safe for use in occupied spaces without fear of oxygen deprivation. Current technology for the manufacture of HFC-236fa is to react 1,1,1,3,3,3-hexachloropropane (HCC-230fa) with anhydrous HF. HCC-230fa has to be made separately. Current technology for the manufacture of HFC-227ea is to react hexafluoropropene (HFP) with hydrogen fluoride (HF). For example, U.S. Pat. No. 5,689,019 describes a method for manufacturing HFC-227ea wherein hexafluoropropene is reacted with anhydrous hydrogen fluoride under the presence of an antimony catalyst. Additionally, U.S. Pat. No. 6,281,395 teaches a method for manufacturing HFC-227ea wherein hexafluoropropene is contacted in the vapor phase at a temperature of less than 260° C. with hydrogen fluoride in the presence of a fluorination catalyst to produce a product. This product is then treated to remove perfluorobutylene (i.e.(CF₃)₂C═CF₂). However, processes which utilize HFP are undesirable for two reasons. First, the preparation of HFP typically involves high reaction temperatures that lead to the formation of extremely toxic by-products, such as perfluorobutylene. Second, because of the high cost involved with removing and destroying such by-products, the raw material cost of HFP is very high. As a result, the cost of HFC-227ea is also very high.

Accordingly, alternate methods of manufacturing HFC-227ea are desired. Suitable alternate techniques for forming HFC-227ea are known in the art. For example, U.S. Pat. No. 5,780,691 teaches a process in which HFC-227ea is formed wherein 1,1,1,3,3,3-hexafluoropropane (HFC-236fa) is reacted with elemental fluorine in an inert gas environment. Further, U.S. Pat. No. 6,191,326 teaches a process for forming HFC-227ea in which 2-trifluoromethyl-3,3,3-trifluoropropionic acid is reacted with fluorine gas.

The present invention provides another alternative solution. The inventive process is able to convert any mixture of three-carbon hydrocarbon (HC), hydrochlorofluorocarbon (HCFC), chlorofluorocarbon (CFC) compounds or halogenated propanes and produce high valued three-carbon hydrofluorocarbons (HFCs) at significantly lower cost than current commercial processes. The methods of this invention include a chloro-fluorination treatment of a three-carbon feed material, forming intermediate products of 2,2-dichloro-1,1,1,3,3,3-hexafluoropropane (CFC-216aa) and 2-chloro-1,1,1,2,3,3,3-heptafluoropropane (CFC-217ba), followed by hydrogenation of these intermediate products to form HFC-236fa and HFC-227ea respectively. Suitable three-carbon feed materials include three-carbon alkanes, alkenes, halo-alkanes and halo-alkenes, as well as halogenated waste product mixtures produced from commercial processes that manufacture three-carbon compounds. Accordingly, the invention provides for a two-step process to manufacture valuable products from low value raw materials including by-products and/or waste streams that are normally incinerated.

### DESCRIPTION OF THE INVENTION

The invention provides a process for the manufacture of hydrofluorocarbons or hydrochlorofluorocarbons comprising:
a) reacting a mixture of at least two three-carbon reactants with an effective amount of chlorine and hydrogen fluoride in a reactor in the vapor phase and in the presence of a fluorination catalyst to form a product which comprises a mixture of 2,2-dichloro-1,1,1,3,3,3-hexafluoropropane and 2-chloro-1,1,1,2,3,3,3-heptafluoropropane; and then
b) separating at least one of 2,2-dichloro-1,1,1,3,3,3-hexafluoropropane and/or 2-chloro-1,1,1,2,3,3,3-heptafluoropropane from said mixture.

The invention also provides a process for the manufacture of hydrofluorocarbons or hydrochlorofluorocarbons comprising:
a) reacting a mixture of at least two three-carbon reactants with an effective amount of chlorine and hydrogen fluoride in a reactor in the vapor phase and in the presence of a fluorination catalyst to form a product which comprises a mixture of 2,2-dichloro-1,1,1,3,3,3-hexafluoropropane and 2-chloro-1,1,1,2,3,3,3-heptafluoropropane; and then
b) separating at least one of 2,2-dichloro-1,1,1,3,3,3-hexafluoropropane and/or 2-chloro-1,1,1,2,3,3,3-heptafluoropropane from said mixture; and then
c) reacting said separated 2,2-dichloro-1,1,1,3,3,3-hexafluoropropane and/or 2-chloro-1,1,1,2,3,3,3-heptafluoropropane with hydrogen under conditions sufficient to form a reaction product which comprises 1,1,1,3,3,3-hexafluoropropane, or 1,1,1,2,3,3,3-heptafluoropropane or mixtures thereof.

In the process of the invention, a mixture of three-carbon reactant feed material is subjected to a chlorination reaction and a fluorination reaction to form an intermediate product which comprises 2,2-dichloro-1,1,1,3,3,3-hexafluoropropane (CFC-216aa) and/or 2-chloro-1,1,1,2,3,3,3-heptafluoropropane (CFC-217ba). In the preferred embodiment, the mixture of three-carbon reactants is simultaneously reacted chlorine and hydrogen fluoride. In another embodiment, the mixture of three-carbon reactants is first reacted with chlorine and then reacted with hydrogen fluoride. In yet another embodiment, the mixture of three-carbon reactants is first reacted with hydrogen fluoride and then reacted with chlorine if unreacted three-carbon reactants, hydrogen fluoride and chlorine are recycled back to step (a). The three-carbon feed material may be any mixture of three-carbon hydrocarbons, three-carbon hydrochlorofluorocarbons, three-carbon chlorofluorocarbons and combinations thereof and are readily available as halogenated waste product mixtures produced from any commercial process that manufactures such three-carbon compounds. Examples of these commercial processes include, but is not limited to, process for forming 1,1,1,3,3,3-hexachloropropane (HCC-230fa), 1,1,1,3,3-pentachloropropane (HCC-240fa), 1,1,1,3-tetrachloropropane (HCC-250fb), 1,1,1-trifluoropropene (TFP), 1,1,1,3,3,3-hexafluoropropane (HFC-236fa) and 1,1,1,3,3-pentafluoropropane (HFC-245fa). Preferred three-carbon reactants include propane, propene, halogenated propanes and halogenated propenes. More specifically, such three-carbon reactants preferably include 1,1-dichloro-3,3,3-trifluoropropane (HCFC-243fa), 1,2,2-trichloro-1,3,3,3-tetrafluoropropane (HCFC-224aa), 1-chloro-1,3,3,3-tetrafluoropropane (HCFC-244fa), 1,2-dichloro-3,3,3-trifluoropropene (HCFC-1223xd), dichlorotetrafluoropropanes (HCFC-234).

The first step of the process involves the chlorination and fluorination of the three-carbon reactant mixture. According to the process of the invention, the three-carbon mixture feed material is fed into a reactor either in the vapor phase or the feed material may be vaporized once inside the reactor. The feed material is then subjected to a chlorination reaction by reacting the feed material with an effective amount of elemental chlorine in the vapor phase. The three-carbon feed material is subjected to a fluorination reaction by reacting the feed material mixture with hydrogen fluoride in the reactor in the vapor phase and in the presence of a fluorination catalyst. Similar to the feed material, the hydrogen fluoride reactant may either be pre-vaporized or vaporized in the reactor.

Suitable fluorination catalysts include transition metal halides and oxides, Group IVb metal halides and oxides, Group Vb metal halides and oxides and combinations thereof. The fluorination catalyst may also comprise a catalyst selected from the group consisting of Cr₂O₃, Cr₂O₃/Al₂O₃, Cr₂O₃/AlF₃, Cr₂O₃/carbon, CoCl₂/Cr₂O₃/Al₂O₃, NiCl₂/Cr₂O₃/Al₂O₃, CoCl₂/AlF₃, and NiCl₂/AlF₃. The preferred fluorination catalyst is chromium (III) oxide. In the preferred process of the invention, the reactor is pre-charged with the fluorination catalyst prior to feeding the reactants into the reactor.

Additionally, the catalyst may be pre-treated either chemically and/or physically to create active sites which facilitate fluorination. For example, the catalyst can be pretreated by calcining it under a flow of inert gas, such as nitrogen, at a temperature comparable to or higher than that of the fluorination reaction. Next, the calcined catalyst is exposed to a fluorinating agent alone or in combination with up to about 5 to about 99 weight percent of inert gas at a temperature of from about 25° C. to about 450° C. for at least about one hour.

In the preferred process of the invention, the chlorination and fluorination reaction is preferably conducted with a molar amount of molecular chlorine which ranges from about 1 mole % to about 50 mole % per mole of three-carbon reactant mixture, more preferably from about 2 mole % to about 40 mole % per mole of three-carbon reactant mixture and most preferably from about 3 mole % to about 30 mole % per mole of three-carbon reactant mixture. Further, the chlorination-fluorination reaction is preferably conducted wherein the mole ratio of said three-carbon reactant mixture to hydrogen fluoride is from about 1:2 to about 1:100, more preferably the mole ratio of said three-carbon reactant mixture to hydrogen fluoride is from about 1:3 to about 1:90 and most preferably from about 1:4 to about 1:80.

The temperature at which the vapor-phase chlorination-fluorination reaction is conducted and the period of reaction will depend on the starting materials, amounts used, and the catalyst used. Such parameters would be readily apparent to one skilled in the art. In the formation of CFC-216aa and CFC-217ba, the chlorination and fluorination reactions of the invention are preferably conducted at a temperature of from about 50° C. to about 600° C., more preferably from about 100° C. to about 500° C. and most preferably from about 150° C. to about 450° C. Further, the reactions of the invention are preferably conducted at a pressure of from about 0 psig to about 500 psig, more preferably from about 2 psig to about 400 psig, and most preferably from about 5 psig to about 300 psig. The reactions are also preferably conducted in an inert atmosphere, e.g. a nitrogen atmosphere, that contains no oxygen. The reactants also may be fed individually or as a mixture to the reactor, or diluted with an inert material, e.g. nitrogen or argon, or perhalogenated material. Once the reaction is underway, the reactants may be continuously under pressure to supply the additional amounts of reactants needed to continue the process.

This chlorination-fluorination process yields mainly CFC-216aa and CFC-217ba and various intermediate fluorocarbon and chlorofluorocarbon products that can be recycled to make more CFC-216aa and CFC-217ba. Additional products of the chlorination-fluorination reaction include hydrochloric acid (HCl). Following the formation of intermediate products, the fluorocarbon products are separated from any unreacted HF, any unreacted chlorine, HCl and any other by-products that may be present via distillation. Each of these separated intermediates and any unreacted starting materials, also known as a remainder, may then be recycled for further reaction or use in other applications.

Upon separation of the fluorocarbon and/or chlorofluorocarbon intermediate products, which may be, for example, such as CFC-216aa and 217ba, these materials are then hydrogenated. Hydrogenation is a chemical reaction between molecular hydrogen and a compound in the presence or absence of a catalyst. The reaction is one in which hydrogen abstracts a halogen atom from a carbon atom and replaces it with a hydrogen atom and/or adds to a double or triple bond connecting two carbon atoms in the structure of the molecule. Hydrogenation catalysts are common and well known in the art, but not all hydrogenation reactions require a catalyst. It is preferred not to use a catalyst when it is not required. When catalyst is required, the catalysts preferred for use in the invention include, but are not limited to metals such as cobalt, nickel, molybdenum, chromium, platinum, palladium and their oxides. Other known catalysts including nickel, Raney nickel, copper chromite, palladium black, palladium hydroxide, rhodium, ruthenium and ruthenium chloride are also suitable. Each of these hydrogenation catalysts may be supported or unsupported. Suitable catalyst supports include carbon, silica, alumina, silicon carbide, aluminum fluoride, and calcium fluoride. Preferred supports are alumina or activated carbon. The hydrogen source is typically hydrogen gas, optionally in admixture with a carrier gas that is inert to the process of the invention (e.g., nitrogen or a noble gas such as helium or argon).

The hydrogenation reaction is preferably carried out at a temperature of from about −20° C. to about 700° C., more preferably from about 0° C. to about 500° C. and most preferably from about 25° C. to about 400° C. Additionally, said hydrogenation reaction is preferably carried out for a contact time of from about 0.1 second to about 72 hours, more preferably from about 1 seconds to 36 hours and most preferably from about 10 seconds to about 1 hour.

In the preferred process of the invention, a three-carbon feed material is chosen which comprises materials that result in the formation of 2,2-dichloro-1,1,1,3,3,3-hexafluoropropane (CFC-216aa) and 2-chloro-1,1,1,2,3,3,3-heptafluoropropane (HCFC-217ba) as an intermediate reaction products, subsequent to said chlorination and fluorination reactions. For this purpose, preferred three-carbon compounds include propane, propene, halogenated propanes and halogenated propenes. More specifically, such three-carbon reactants preferably include 1,1-dichloro-3,3,3-trifluoropropane (HCFC-243fa), 1,2,2-trichloro-1,3,3,3-tetrafluoropropane (HCFC-224aa), 1-chloro-1,3,3,3-tetrfluoropropane (HCFC-244fa), 1,2-dichloro-3,3,3-trifluoropropene (HCFC-1223xd) and dichlorotetrafluoropropane (HCFC-234). These intermediate materials are then separated from unreacted starting materials and by-products and 2,2-dichloro-1,1,1,3,3,3-hexafluoropropane and 1,1,1,2,3,3,3-heptafluoropropane are recovered. Subsequently, a hydrogenation reaction is conducted that converts 2,2-dichloro-1,1,1,3,3,3-hexafluoropropane and 2-chloro-1,1,1,2,3,3,3-heptafluoropropane (CFC-217ba) into of 1,1,1,3,3,3-hexafluoropropane (HFC-236fa) and 1,1,1,2,3,3,3-heptafluoropropane(HFC-227ea), respectively.

The following non-limiting examples serve to illustrate the invention. It will be appreciated that variations in proportions will be apparent to those skilled in the art and are within the scope of the present invention.

### EXAMPLE 1

At 400° C., 39 g/hr of mixed 3-carbon HCFC's, anhydrous hydrofluoric acid at 5:1 mole ratio and chlorine at 2:1 mole ratio are co-fed to a vapor phase reactor containing a chrome oxide catalyst. In a single pass, 95% of the feed materials are reacted to produce 79% CFC-216aa and 10% CFC-217ba with the balance consisting of other CFC-216 and CFC-217 isomers and some CFC-215 isomers. The feed material consisted of approximately 40% HCFC-1223xd, 33% HCFC-243fa, 17%, HCFC-224aa, with the balance being other three carbon HCFC compounds.

### EXAMPLE 2

CFC-216aa and hydrogen gas are fed to a 1″×32″ Monel reactor. The reactor is packed with nickel mesh and heated to 400° C. at 500 psig. CFC-216aa is fed at 0.5 g/min and hydrogen is fed at a rate that gives 20/1 mole ratio. No catalyst is used in this reaction. About 60 GC area % of CFC-216aa is converted and selectivity of HFC-236fa is about 95%.

### EXAMPLE 3

In the same equipment as described in Example 2, a catalyst of palladium metal supported on activated carbon (100 ml) is used as catalyst. The reaction temperature is at 250° C. and pressure is at 200 psig. Conversion of CFC-216aa is about 90% and selectivity of HFC-236fa is about 97%.

### EXAMPLE 4

CFC-217ba and hydrogen gas are fed to a 1″×32″ Monel reactor. The reactor is packed with nickel mesh and heated to 400° C. at 500 psig. CFC-217ba is fed at 0.5 g/min and hydrogen is fed at a rate that gives 20/1 mole ratio. No catalyst is used in this reaction. About 80 GC area % of CFC-217ba is converted and selectivity of HFC-227ea is about 90%.

### EXAMPLE 5

In the same equipment as described in Example 4, a catalyst of platinum oxide supported on alumina (100 ml) is used as catalyst. The reaction temperature is at 250° C. and pressure is at 200 psig. Conversion of CFC-217ba is about 90% and selectivity of HFC-227ea is about 95%.

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

1. A process for the manufacture of hydrofluorocarbons or hydrochlorofluorocarbons comprising:
a) reacting a mixture of at least two three-carbon reactants with an effective amount of chlorine and hydrogen fluoride in a reactor in the vapor phase and in the presence of a fluorination catalyst to form a product which comprises a mixture of 2,2-dichloro-1,1,1,3,3,3-hexafluoropropane and 2-chloro-1,1,1,2,3,3,3-heptafluoropropane; and then
b) separating at least one of 2,2-dichloro-1,1,1,3,3,3-hexafluoropropane and/or 2-chloro-1,1,1,2,3,3,3-heptafluoropropane from said mixture.

2. The process of claim 1 wherein 2,2-dichloro-1,1,1,3,3,3-hexafluoropropane is separated from said mixture.

3. The process of claim 1 wherein 2-chloro-1,1,1,2,3,3,3-heptafluoropropane is separated from said mixture.

4. The process of claim 1 further comprising the subsequent step of:
c) reacting said separated 2,2-dichloro-1,1,1,3,3,3-hexafluoropropane and/or 2-chloro-1,1,1,2,3,3,3-heptafluoropropane with hydrogen under conditions sufficient to form a reaction product which comprises 1,1,1,3,3,3-hexafluoropropane, or 1,1,1,2,3,3,3-heptafluoropropane or mixtures thereof.

5. The process of claim 1 wherein 2,2-dichloro-1,1,1,3,3,3-hexafluoropropane is separated from said mixture, and further comprising reacting said 2,2-dichloro-1,1,1,3,3,3-hexafluoropropane with hydrogen under conditions sufficient to form a reaction product which comprises 1,1,1,3,3,3-hexafluoropropane.

6. The process of claim 1 wherein 2-chloro-1,1,1,2,3,3,3-heptafluoropropane is separated from said mixture, and further comprising reacting said 2-chloro-1,1,1,2,3,3,3-heptafluoropropane with hydrogen under conditions sufficient to form a reaction product which comprises 1,1,1,2,3,3,3-heptafluoropropane.

7. The process of claim 1 wherein each of 2,2-dichloro-1,1,1,3,3,3-hexafluoropropane and 2-chloro-1,1,1,2,3,3,3-heptafluoropropane are separated from said mixture, and further comprising reacting each of said separated 2,2-dichloro-1,1,1,3,3,3-hexafluoropropane and 2-chloro-1,1,1,2,3,3,3-heptafluoropropane with hydrogen under conditions sufficient to form 1,1,1,3,3,3-hexafluoropropane and 1,1,1,2,3,3,3-heptafluoropropane.

8. The process of claim 1 wherein said three-carbon reactants comprise a material selected from the group consisting of three-carbon hydrocarbons, three-carbon hydrochlorofluorocarbons, three-carbon chlorofluorocarbons and combinations thereof.

9. The process of claim 1 wherein said three-carbon reactants comprise a material selected from the group consisting of propane, propene, halogenated propanes, and halogenated propylene.

10. The process of claim 4 wherein said reaction with hydrogen is conducted without use of a catalyst.

11. The process of claim 4 wherein said reaction with hydrogen is conducted by using of a hydrogenation catalyst.

12. The process of claim 11 wherein said hydrogenation catalyst comprises palladium, palladium black, palladium hydroxide, palladium oxides, platinum, platinum oxides, nickel, Raney nickel, ruthenium chloride, ruthenium, rhodium, cobalt, molybdenum, chromium, copper chromite or combinations thereof.

13. The process of claim 11 wherein said hydrogenation catalyst comprises alumina or activated carbon supported palladium, palladium black, palladium hydroxide, palladium oxides, platinum oxides, nickel, Raney nickel, ruthenium chloride, ruthenium, rhodium, cobalt, molybdenum, chromium, platinum, copper chromite or combinations thereof.

14. The process of claim 1 wherein said fluorination catalyst is selected from the group consisting of transition metal halides and oxides, Group IVb metal halides and oxides, Group Vb metal halides and oxides and combinations thereof.

15. The process of claim 1 wherein said fluorination catalyst is selected from the group consisting of Cr₂O₃, Cr₂O₃/Al₂O₃, Cr₂O₃/AlF₃, Cr₂O₃/carbon CoCl₂/Cr₂O₃/Al₂O₃, NiCl₂/Cr₂O₃/Al₂O₃, CoCl₂/AlF₃, and NiCl₂/Alf₃.

16. The process of claim 1 wherein said fluorination catalyst comprises chromium (III) oxide.

17. The process of claim 1 wherein said hydrogen fluoride is pre-vaporized.

18. The process of claim 1 wherein said reacting is conducted at a temperature of from about 150° C. to about 450° C.

19. The process of claim 1 wherein said reacting is conducted at a pressure of from about 5 psig to about 300 psig.

20. The process of claim 1 wherein said reacting is conducted at atmospheric pressure.

21. The process of claim 1 wherein said reaction is conducted with a molar amount of chlorine which ranges from about 3 mole % to about 30 mole % per mole of three-carbon reactant mixture.

22. The process of claim 1 wherein the mole ratio of said mixture of three-carbon reactants to hydrogen fluoride is from about 1:2 to about 1:100.

23. The process of claim 1 wherein the mole ratio of said mixture of three-carbon reactants to hydrogen fluoride is from about 1:4 to about 1:80.

24. The process of claim 1 wherein the result from step (b) produces a remainder and recycling the remainder by adding it to the mixture of step (a).

25. The process of claim 4 wherein said reacting with hydrogen step is carried out at a temperature of from about −20° C. to about 700° C.

26. The process of claim 4 wherein said reacting with hydrogen step is carried out for a time of from about 0.1 seconds to about 72 hours.
